(19) United States
(12) Patent Application Publication
(10) Pub. No.: US 2011/0206775 A1
(43) Pub. Date: Aug. 25, 2011

(54) Preparation of inert pharmaceutical excipients for stabilizing unstable pharmaceutical ingredients

(76) Inventor: Pieter J. Groenewoud, Powder Springs, GA (US)

(21) Appl. No.: 13/015,228

(22) Filed: Jan. 27, 2011

Related U.S. Application Data
(60) Provisional application No. 61/298,610, filed on Jan. 27, 2010.

(51) Int. Cl. A61K 31/198; C08B 1/00; A61K 31/137; A61K 35/55; A61P 5/14

(52) U.S. Cl. 424/568; 536/56; 514/567; 514/653; 514/649

(57) ABSTRACT

In a method of treating a pharmaceutical component, a reducing substance is added to a solution of water and a pharmaceutical excipient having at least one residual reactive impurity. The reducing substance is allowed to react with the residual reactive impurity, thereby generating an inert reaction product. The any remaining amount of the reducing substance is removed from the pharmaceutical excipient. The pharmaceutical excipient is dried after the removing step.

### CROSS-REFERENCE TO RELATED APPLICATION

This application claims the benefit of U.S. Provisional Patent Application Ser. No. 61/298,610, filed Jan. 27, 2010, the entirety of which is hereby incorporated herein by reference.

### BACKGROUND OF THE INVENTION

1. Field of the Invention

The present invention relates to pharmaceuticals and, more specifically, to a system for stabilizing unstable pharmaceuticals.

2. Description of the Related Art

Many active pharmaceutical ingredients (API's) do not behave inert in a finished product matrix such as a tablet or capsules. Some API's are inherently unstable and when combined with other ingredients for pharmaceutical processing they exhibit significant degradation. Sometimes when the degradation products are pharmacologically active in a similar manner as the API, it may present less of a problem because the overall potency of the drug is not measurably affected. However, when the potency is affected, therapeutic effectiveness can be affected as well. Examples of this are thyroxin medications such as levothyroxine, liothyronine, thyroid extract, dextrothryroxine, triiodophenylethylamine etc. Furthermore thyroxin medications are classified as narrow therapeutic index (NTI) compounds that require careful titration for every individual patient. Although there have been several attempts to improve their stability, finished pharmaceutical products that contain thyroxin medications still have insufficient stability or contain stabilizers that present potential problems for patients relying on such therapies.

In the case of insufficient stability, potency decreases over its shelf life and can under-medicate patients or interfere with accurate titration by physicians. In case of presence of stabilizers, such as potassium or sodium iodide, the FDA has cautioned physicians about the use of these medications because higher levels of iodides are found in natural sources such as drinking water in certain geographical areas. This restricts the use of these stabilized thyroxin medications in these areas. In addition, taking iodine containing formulas in combination with multivitamin/mineral supplements could lead to too high levels of iodine in the body by exceeding the recommended daily allowance, thus discouraging physicians to prescribe this more stable dosage form.

Pharmaceutical finished dosage forms are almost all combinations of API's and excipients. The excipients are necessary to transform API's into dosage forms that can be self administered by patients. Most dosage forms are in the form of tablets or capsules and these dosage forms in particular are manufactured with the help of excipients. An ideal excipient has effective functionally, but does not adversely affect the stability of the API in the finished dosage form at the level that it is typically applied. Excipients are used as binders, disintegrants, glidants, carriers, diluents, bulking agents, etc. One excipient group that is particularly useful includes derivatives of cellulose. Because of its unique chemical structure, cellulose is known for its stability and relative inertness compared to other excipients making it less likely to cause degradation of the API. Examples of processed cellulose include: powdered cellulose, microcrystalline cellulose (mcc), silicified microcrystalline cellulose, hydroxypropylmethyl cellulose, hydroxyethylcellulose, hydroxypropyl cellulose, ethyl cellulose, carboxymethyl cellulose and its salts, cellulose acetate, methyl cellulose etc.

However, while cellulose is relatively stable and inert, extensive processing of cellulose is needed to yield pharmaceutical grade products and can introduce chemicals that are difficult to be completely removed or inactivated. These compounds include, but are not limited to, sodium hydroxide, bleaching agents, propylene oxide, ethylene oxide etc. In most cases, these trace impurities do not interfere with the stability of an API, however when very low amounts (sometimes well below 1 mg per dose) of API are used, the ratio of the impurities to API becomes much higher and can have an impact on product stability.

Therefore, there is a need for a method to process cellulose derived excipients so as render impurities inert.

### SUMMARY OF THE INVENTION

The disadvantages of the prior art are overcome by the present invention which, in one aspect, is a method of treating a pharmaceutical component, in which a reducing substance is added to a pharmaceutical excipient having at least one residual reactive impurity. The reducing substance is allowed to react with the residual reactive impurity, thereby generating an inert reaction product. The any remaining amount of the reducing substance is removed from the pharmaceutical excipient.

In another aspect, the invention is a method of generating a pharmaceutical composition, in which a predetermined amount of a reducing substance is dissolved into water, thereby forming a solution. A cellulose-derived excipient is mixed with the solution, thereby forming a suspension so that the reducing substance reacts with at least one residual reactive impurity in the cellulose-derived excipient so as to generate inert reaction products. The suspension is filtered after a predetermined amount of time, thereby forming a cake. The cake is washed with water thereby removing the reducing substance. The cellulose-derived excipient is added to an unstable active pharmaceutical ingredient, thereby forming the pharmaceutical composition.

In another aspect, the invention is a method of generating a thyroxin pharmaceutical composition, in which a predetermined amount of an iodide is dissolved into water, thereby forming a solution. A technical grade of a cellulose-derived excipient is mixed with the solution, thereby forming a suspension. Impurities in the cellulose-derived excipient are allowed to react with the iodide for a predetermined amount of time so as to form inert reaction products. The suspension is filtered after the predetermined amount of time, thereby forming a cake. The cake is washed with water thereby removing iodide therefrom. The cellulose-derived excipient is dried and milled after the washing step, thereby forming a dried cellulose-derived excipient. The dried cellulose-derived excipient is added to a thyroxin medication thereby forming the thyroxin pharmaceutical composition.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

A general disclosure of stabilized thyroxin medications is presented in U.S. Pat. No. 6,190,696, issued to Groenewoud, which is incorporated by reference herein.

One embodiment uses iodine and other stabilizers to treat excipients, letting them react with traces of processing chemicals, which are subsequently removed before the medication is processed into a finished dosage form. This way the finished product does not contain undesirable additives yet yields a stable product. In one embodiment, pharmaceutical grade cellulose and cellulose derivatives (collectively referred to herein as “cellulose-derived substances”) are processed so as to yield a grade that is essentially chemically inert without affecting its physical and pharmaceutical functionality, allowing for the production of stable dosage forms that otherwise would exhibit chemical instability. Examples of cellulose-derived substances include: microcrystalline cellulose, silicified microcrystalline cellulose, powdered cellulose, hydroxypropylmethylcellulose, hydroxycellulose, hydroxypropylcellulose, ethyl cellulose, cellulose acetate, carboxymetylcellulose, cellulose acetate phthalate, hydroxyethylpropyl cellulose, hypromellose acetate succinate, hypromellose phthalate, methyl cellulose, and combinations thereof.

One embodiment of the invention employs potassium, calcium, sodium iodides or other acceptable sources of iodide, antioxidants such as ascorbic acid, butylated hydroxyanisole and any other suitable reducing agents. (Examples of iodides include: sodium iodide, potassium iodide, calcium iodide, magnesium iodide, butylhydroxytoluene, butylhydroxyanisole, ascorbic acid, ascorbyl palmitate, potassium metabisulfite, a metabisulfite salt, sodium sulfite, ant sulfite salt, and combinations thereof.) Water soluble antioxidants are particularly useful because they do not require the use of more expensive solvents such as alcohols etc. These substances are first introduced to react with residual impurities in the cellulose substances and are subsequently removed to yield excipients that can be processed with unstable active ingredients allowing for improved stability without the presence of undesirable compounds such as iodides. The resulting inert excipient may be combined with an unstable active ingredient, such as a thyroxin medication. (Examples of thyroxin medications include: levothyroxine, liothyronine, thyroid extract, dextrothryroxine, triiodophenylethylamine, and combinations thereof.)

In one embodiment, water soluble iodide salts are used since they can be used to stabilize thyroxin dosage forms. One possible reason for this is that iodides are relatively mild reducing agents. Since some molecules of API's may have functional groups that are sensitive to reducing as well as oxidizing substances, it is important to choose antioxidants that do not cause degradation by themselves because of their reducing nature.

In one example, a solution of 0.2% sodium iodide is prepared by dissolving 200 g sodium iodide in 100 L purified water. Once a clear solution is obtained 10 kg of microcrystalline cellulose (MCC) such as Avicel®, is added to form a suspension. The MCC is allowed to react for a period of several hours before it is filtered. The filtered cake is washed with purified or distilled water to remove the sodium iodide and is subsequently dried and milled (or screened) to obtain MCC that is similar in appearance as the starting material but is rendered inert because trace amounts of residual oxidative processing compounds have been inactivated. The MCC is now ready for further processing into finished dosage forms with unstable API's such as Levothyroxine, Liothyronine or in a combination.

A solution 0.2% ascorbic acid is prepared by dissolving 200 g Ascorbic Acid in 100 L of water. Once dissolved 10 kg of powdered cellulose such as Elcema® is added and allowed to react for several hours under slow stirring. The material is filtered, washed with purified or distilled water until the water has a neutral pH. The material is filtered again, dried and screened to obtain inert powdered cellulose. The material can be stored or used for further processing with unstable API's that require an inert finished product matrix.

In another embodiment Sodium Iodide is dissolved in 1.5 L purified or distilled water and dispersed over 10 kg of Avicel® (ph101, 102 or other technical grades) in a high shear granulator. The damp material is dried in a tray during oven or fluid bed dryer to about +/−1% from the original moisture content. The dried material is further processed by washing with distilled water to remove the iodide, filtered and dried as described earlier.

In another embodiment 15 g of sodium iodide is dissolved in 500 mL of alcohol. The alcoholic solution is dispersed over 5 kg of hydroxypropylmethylcellulose (any technical grade or brand) by the use of a high shear granulator. The material is dried using a fluid bed dryer or other suitable drying process such as vacuum drying. The material is subsequently washed with alcohol and then filtered or centrifuged. The resulting cake is tested for the absence of iodide and dried and milled to yield material that is suitable for further processing into pharmaceutical finished dosage forms or used for coating of tablets.

In another embodiment 10 g butylated hydroxytoluene (BHT) is dissolved in 1.5 L of alcohol or acetone. The solution is dispersed over 10 kg of any technical grade of silicified microcrystalline cellulose (ProSolv®) with the use of suitable granulating equipment and subsequently dried until all solvent is removed. The resulting agglomerated dry material is washed with the same solvent as used for the dispersion of the BHT solution and filtered and dried. The dried material is sieved and/or milled to yield material that is similar in appearance as the staring material but rendered chemically inert through the effective removal of residual oxidative substances in the starting material, yet has the same pharmaceutical processing characteristics as the original material.

In another embodiment a 0.1% solution of ascorbic acid is prepared by dissolving 10 g in 10 L of purified water. This solution is used to react with residual impurities in water insoluble cellulose derivatives such powdered cellulose, microcrystalline cellulose, silicified microcrystalline cellulose, ethyl cellulose etc. The material is added to the solution in an amount sufficient to yield thin slurry. The solution is allowed to react with the cellulose derivative for several hours before it is washed, dried and milled to its original particle size specifications.

In another embodiment ascorbyl palmitate is used as the reactant to remove reactive trace impurities in processed cellulose derivatives. It is first dissolved in alcohol or other suitable solvent, to a level of 0.05% or higher. This solution is used to react with cellulose derivatives that cannot be neutralized in an aqueous medium because of their water solubility and therefore would create a solution with high viscosity that is difficult to further process. Cellulose derivatives are added in a sufficient quantity to yield a thin slurry and are allowed to react for several hours. The material is then washed and processed as described earlier.

The purified materials that are obtained this way can now be processed into pharmaceutical dosage forms such as tablets or capsules as follows.

### Example 1

| | | | | Per 100,000 | | |
|---|---|---|---|---|---|---|
| | Ingredient | Per Tab | | tablets | | |
| 1 | Thyroid Extract | 120.00 | mg | 12.00 | kg | 24.00% |
| 2 | Microcrystalline Cellulose | 147.45 | mg | 14.75 | kg | 29.49% |
| | treated with Ascorbyl | | | | | |
| | Palmitate | | | | | |
| 3 | Sodium Starch Glycolate | 5.00 | mg | 500.00 | g | 1.00% |
| 4 | Magnesium Stearate | 2.50 | mg | 250.00 | g | 0.50% |

The materials 1, 2 and 3 are screened through a 40 mesh sieve and blended for about twenty minutes in a V-blender. Material 4 is added and the blending is continued for 3 more minutes. The powder can now be compressed into tablets weighing 500 mg each and be stored at room temperature without significant degradation. Alternatively the powder can also be filled into hard gelatin capsules or HPMC capsules.

### Example 2

Batch size 100,000 tablets

| | | Per tablet | | Per batch | |
|---|---|---|---|---|---|
| 1. | Liothyronine sodium | 0.05 | mg | 5.00 | g |
| 2. | Microcrystalline cellulose treated with | 98.45 | mg | 9845 | g |
| | Potassium Iodide | | | | |
| 3. | Sodium starch glycolate | 1.00 | mg | 100 | g |
| 4. | Magnesium Stearate | 0.50 | mg | 50 | g |
| | Total: | 100 | mg | 10.00 | kg |

Material 1. is intensively blended with part of material 2 through geometrical dilutions. This is now further blended with the rest of material 2. and material 3. for 20 minutes in a V-blender or other suitable blender such as double cone blender. After 20 minutes the magnesium stearate can be added and blended for an additional 3 minutes. The resulting powder is now ready to be compressed into tablets each weighing 100 mg.

### Example 3

Batch size 100,000 tablets

| | | Per tablet | | Per batch | |
|---|---|---|---|---|---|
| 1. | Levothyroxine sodium | 0.30 | mg | 30.00 | g |
| 2. | Microcrystalline cellulose treated with | 98.45 | mg | 9845 | g |
| | Ascorbic acid | | | | |
| 3. | Sodium starch glycolate | 1.00 | mg | 100 | g |
| 4. | Magnesium Stearate | 0.50 | mg | 50 | g |
| | Total: | 100 | mg | 10.00 | kg |

Material 1. is intensively blended with part of material 2 through geometrical dilutions. This is now further blended with the rest of material 2. and material 3. for 20 minutes in a V-blender or other suitable blender such as double cone blender. After 20 minutes the magnesium stearate can be added and blended for an additional 3 minutes. The resulting powder is now ready to be compressed into tablets each weighing 100 mg.

### Example 4

Batch size 100,000 tablets

| | | Per tablet | | Per batch | |
|---|---|---|---|---|---|
| 1. | Phenylephrine | 60.0 | mg | 6000 | g |
| 2. | Microcrystalline cellulose treated with | 186.25 | mg | 18.63 | kg |
| | Butylated Hydroxytoluene | | | | |
| 3. | Sodium starch glycolate | 2.50 | mg | 250.00 | g |
| 4. | Magnesium Stearate | 1.25 | mg | 125.00 | g |
| | Total: | 250 | mg | 25.00 | kg |

The materials 1, 2 and 3 are screened through a 40 mesh sieve and blended for about twenty minutes in a V-blender. Material 4 is added and the blending is continued for 3 more minutes. The powder can now be compressed into tablets weighing 500 mg each and be stored at room temperature without significant degradation. Alternatively the powder can also be filled into hard gelatin capsules or HPMC capsules.

### Example 5

Batch size 100,000 tablets

| | | Per tablet | | Per batch | |
|---|---|---|---|---|---|
| 1. | Triiodophenylethylamine | 0.03 | mg | 3.00 | g |
| 2. | Microcrystalline cellulose treated with | 246.22 | mg | 24.62 | kg |
| | Potassium Iodide | | | | |
| 3. | Sodium starch glycolate | 2.50 | mg | 250.00 | g |
| 4. | Magnesium Stearate | 1.25 | mg | 125.00 | g |
| | Total: | 250 | mg | 25.00 | kg |

Material 1 is intensively blended with part of material 2 through geometrical dilutions. This is now further blended with the rest of material 2 and material 3 for 20 minutes in a V-blender or other suitable blender such as double cone blender. After 20 minutes the magnesium stearate can be added and blended for an additional 3 minutes. The resulting powder is now ready to be compressed into tablets each weighing 250 mg.

Reducing substances may be used to treat widely used pharmaceutical excipients to convert these into the chemically most inert form possible. This is accomplished by allowing reducing substances to react with residual processing impurities and other contaminants that have the potential to adversely affect the potency of pharmaceutically active ingredients. The reducing substances are substances that render pharmaceutical excipient completely inert. These substances are removed from the excipients before they are further processed. Especially in the case of the use of iodides, this provides a distinct advantage, in that the medicaments can now be administered without the risk that patients will be exposed to high doses of iodides that exceed the recommended daily allowance.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A method of treating a pharmaceutical component, comprising the steps of:
a. adding a reducing substance to a pharmaceutical excipient having at least one residual reactive impurity;
b. allowing the reducing substance to react with the residual reactive impurity, thereby generating an inert reaction product; and
c. removing any remaining amount of the reducing substance from the pharmaceutical excipient.

2. The method of claim 1, wherein the pharmaceutical excipient includes a technical grade of a cellulose-derived substance.

3. The method of claim 2, wherein the technical grade of the cellulose-derived substance is selected from a group consisting of: microcrystalline cellulose, silicified microcrystalline cellulose, powdered cellulose, hydroxypropylmethylcellulose, hydroxycellulose, hydroxypropylcellulose, ethyl cellulose, cellulose acetate, carboxymetylcellulose, cellulose acetate phthalate, hydroxyethylpropyl cellulose, hypromellose acetate succinate, hypromellose phthalate, methyl cellulose, and combinations thereof.

4. The method of claim 1, wherein the reducing substance comprises an antioxidant.

5. The method of claim 4, wherein the antioxidant is selected from a group consisting of: sodium iodide, potassium iodide, calcium iodide, magnesium iodide, butylhydroxytoluene, butylhydroxyanisole, ascorbic acid, ascorbyl palmitate, potassium metabisulfite, a metabisulfite salt, sodium sulfite, ant sulfite salt, and combinations thereof.

6. The method of claim 1, wherein the reducing substance produces iodide ions upon addition to the pharmaceutical excipient.

7. The method of claim 1, wherein the reducing substance dissolved in water to form a solution, and wherein the solution is mixed with the pharmaceutical excipient so as to form a suspension, and wherein the removing step comprises the actions of:
a. filtering the suspension to form a cake; and
b. washing the cake to dissolve the reaction product and the remaining reaction substance therefrom, thereby forming a purified cake.

8. The method of claim 7, further comprising the step of adding an unstable active pharmaceutical ingredient to the pharmaceutical excipient, thereby forming a pharmaceutical composition.

9. The method of claim 8, wherein the unstable active pharmaceutical ingredient comprises a thyroxin medication.

10. The method of claim 9, wherein the thyroxin medication is selected from a group consisting of: levothyroxine, liothyronine, thyroid extract, dextrothryroxine, triiodophenylethylamine, and combinations thereof.

11. A method of generating a pharmaceutical composition, comprising the steps of:
a. dissolving a predetermined amount of a reducing substance into water, thereby forming a solution;
b. mixing a cellulose-derived excipient with the solution, thereby forming a suspension so that the reducing substance reacts with at least one residual reactive impurity in the cellulose-derived excipient so as to generate intert reaction products;
c. filtering the suspension after a predetermined amount of time, thereby forming a cake;
d. washing the cake with water thereby removing the reducing substance; and
e. adding the cellulose-derived excipient to an unstable active pharmaceutical ingredient, thereby forming the pharmaceutical composition.

12. The method of claim 11, further comprising the steps of:
a. drying the cellulose-derived excipient, thereby generating a dry cellulose-derived excipient prior to the step of adding the cellulose-derived excipient to an unstable active pharmaceutical ingredient; and
b. milling the dry cellulose-derived excipient

13. The method of claim 11, wherein the cellulose-derived excipient includes a technical grade of a cellulose-derived substance.

14. The method of claim 13, wherein the technical grade of the cellulose-derived substance is selected from a group consisting of: microcrystalline cellulose, silicified microcrystalline cellulose, powdered cellulose, hydroxypropylmethylcellulose, hydroxycellulose, hydroxypropylcellulose, ethyl cellulose, cellulose acetate, carboxymetylcellulose, cellulose acetate phthalate, hydroxyethylpropyl cellulose, hypromellose acetate succinate, hypromellose phthalate, methyl cellulose, and combinations thereof.

15. The method of claim 11, wherein the reducing substance comprises an antioxidant.

16. The method of claim 15, wherein the antioxidant is selected from a group consisting of: sodium iodide, potassium iodide, calcium iodide, magnesium iodide, butylhydroxytoluene, butylhydroxyanisole, ascorbic acid, ascorbyl palmitate, potassium metabisulfite, a metabisulfite salt, sodium sulfite, ant sulfite salt, and combinations thereof.

17. The method of claim 11, wherein the reducing substance produces iodide ions upon being dissolved in water.

18. The method of claim 11, wherein the unstable active pharmaceutical ingredient comprises a thyroxin medication.

19. The method of claim 18, wherein the thyroxin medication is selected from a group consisting of: levothyroxine, liothyronine, thyroid extract, dextrothryroxine, triiodophenylethylamine, and combinations thereof.

20. A method of generating a thyroxin pharmaceutical composition, comprising the steps of:
a. dissolving a predetermined amount of an iodide into water, thereby forming a solution;
b. mixing a technical grade of a cellulose-derived excipient with the solution, thereby forming a suspension;
c. allowing, for a predetermined amount of time, impurities in the cellulose-derived excipient to react with the iodide so as to form inert reaction products;
d. filtering the suspension after the predetermined amount of time, thereby forming a cake;
e. washing the cake with water thereby removing iodide therefrom;
f. drying and milling the cellulose-derived excipient after the washing step, thereby forming a dried cellulose-derived excipient; and
g. adding the dried cellulose-derived excipient to a thyroxin medication thereby forming the thyroxin pharmaceutical composition.
